(19) United States
(12) Patent Application Publication
(10) Pub. No.: US 2005/0037409 A1
(43) Pub. Date: Feb. 17, 2005

(54) Translocation tagging

(76) Inventor: Lawrence M. Kauvar, San Francisco, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/903,811

(22) Filed: Jul. 29, 2004

Related U.S. Application Data
(60) Provisional application No. 60/515,295, filed on Oct. 28, 2003.
(60) Provisional application No. 60/490,994, filed on Jul. 29, 2003.

(51) Int. Cl. C12Q 1/68; G01N 33/53; G01N 33/567

(52) U.S. Cl. 435/6; 435/7.2

(57) ABSTRACT

Methods to identify proteins useful as translocation markers employ fusions of these proteins with common epitopes. An improved method for shotgun cloning employs pre-identified particulate labeled members of nucleic acid libraries. Methods to identify proteins useful as translocation markers employ fusions of these pre-identified particulate labeled members of nucleic acid libraries. An improved method for reporter gene assays employs parallel detection of multiple reporters using distinguishable particulate or antigenic labels coupled to specific detection agents for each reporter.

### RELATED APPLICATIONS

The application claims the benefit of priority under 35 U.S.C. 119(e) of U.S. Provisional Patent Application No. 60/515,295, filed Oct. 28, 2003 and U.S. Provisional Patent Application No. 60/490,994, filed Jul. 29, 2003, both of which are hereby incorporated by reference in their entirety.

### TECHNICAL FIELD

The invention is directed to methods to identify and quantify translocating markers that are responsive to altered cellular conditions. The invention also relates to an improved method for shotgun cloning. The identity of the nucleotide sequence that successfully transforms a cell can be ascertained using this method without isolating the insert from that cell. For example, the described invention relates to an improved method to identify translocation markers employing reporter molecules, which can include antigenic markers, the expression of which allows for the detection of transformed cells without requiring the skilled artisan to sequence individual samples. Thus, phenotypically changes such as translocation, which does not give a growth or survival advantage, can be used to select clones of interest.

### BACKGROUND ART

It is recognized generally that alteration of cellular conditions can result in translocation of one or more proteins, often proteins involved in cellular signaling. For example, PCT publication WO 98/45704 utilizes the translocation of a single protein as a method to monitor altered cellular circumstances.

PCT publication WO 00/77517 describes assessing toxicity or disease states of cells by monitoring the effect of a toxin or a disease condition on the location of a multiplicity of intracellular proteins. Methods for analyzing multiple translocation indicators in response to various stimuli are also described in U.S. Pat. No. 6,416,959, incorporated herein by reference, where cells are arrayed and used to screen for the translocation of a marker in response to a multiplicity of conditions each imposed on a well.

Alternative methods to assess microarrays of cells for translocating proteins are described by Camp, R. L., et al., Nature Med. (2002) 8:1323-1327 and in the PCT publication WO 02/086498.

These methods discussed above all use one or more known translocation proteins, which undergo translocation under altered cellular conditions. These methods also follow the movement of these markers by following labels present on the marker proteins as a criterion for response to a particular stimulus.

In spite of the work done regarding the tracking of protein translocation in response to stimuli, there remains a need for a method to identify proteins whose intracellular, membrane surface and/or extracellular locations are responsive to particular changes in defined conditions, such as disease, toxin exposure, and protocols designed to restore normal cellular function.

### SUMMARY OF THE INVENTION

The invention described below related to a systematic approach for identifying proteins that translocate in response to perturbations of cellular conditions. These proteins serve as markers for changes in cellular conditions by virtue of their translocation in response to such perturbations. A number protein types serve as useful marker candidates—for example, protein kinase C (PKC), cAMP-dependent kinase anchoring proteins (AKAP), pleckstrin homology (PH) domain proteins, cytokines and the like. Further, even though some general classes of translocation protein markers are known, the optimal class and the particular members thereof that are useful in response to a particular stimulus are generally not known. The invention method employs a multiplicity of cells transfected with expression vectors for individual proteins wherein the proteins are tagged with an epitope permitting parallel determination of location of these proteins using a single reagent related to the epitope, such as an antibody.

Thus, in one aspect, the invention is directed to a method to identify at least one protein that is translocated in response an externally applied stimulus which method comprises applying said stimulus to a mixture or an array of cells, each cell expressing a candidate marker protein fused to an epitope, wherein said epitope binds to a detection agent common to all said epitopes; wherein the number of cells is large enough so that each candidate from a library is expressed in multiple cells; treating the cells with said detection agent before and after imposing said extracellular stimulus; and comparing the location of each fusion protein in or around individual cells before and after said application; wherein a protein whose location is altered after said application as compared to before said application is identified as a translocation marker for said stimulus.

Translocation may occur intracellularly (e.g., from one cellular compartment to another), from an intracellular compartment to the cell membrane, from the cell membrane to an intracellular compartment, or from an intracellular space or from the cell membrane to an extracellular space (i.e., secreted). Preferably, the proteins are those that would ordinarily be present in the cells.

As the method described above uses a multiplicity of cells altered so as to express a fusion protein, said fusion proteins sharing a common label, the method of the invention can be simplified by transfecting the cells in such a manner that the identity of the transfecting DNA can be ascertained without recovery and sequencing. Thus, in another aspect, the invention is directed to a method to prepare a multiplicity of cells expressing different proteins which method comprises contacting the cells to be transfected with expression vectors coupled to particulate labels wherein each expression vector for an individual protein is labeled with a particulate label of a different hue. The identity of the protein produced by a transfected cell can then be ascertained by reference to the particulate label associated with the expression vector. Suitable, multi-hued particulate labels are known in the art as further described below.

Thus, in one embodiment, the expression vectors comprising the nucleotide sequences encoding the various fusion proteins are constructed from nucleotide sequences that encode pre-identified proteins where the expression vectors are associated with labels, each different nucleotide sequence having its own identifying label. Cells that contain proteins whose presence or translocation has been detected by use of the detection agent then need not be subjected to extraction of the introduced DNA and sequencing, but rather the nature of the protein can be ascertained by the signal from the label with which it is associated.

Alternatively, the nature of the protein expressed can be ascertained by supplying defined cDNAs in a grid pattern identifiable by position, for example each cDNA in a different well of a microplate. A more sophisticated variant of such a system is described by Ziauddin, J., et al., Nature (2001) 411:107-110 which describes electroporating cells lying on a patterned grid of DNA spots such that cells take up DNA locally from the underlying spots.

In a preferred embodiment, the identification of translocation tags can be biased by providing as candidate proteins those most likely to be successful translocation markers. In one preferred embodiment, the candidates are selected among groups known to contain members that exhibit translocation; in another approach, housekeeping genes common to all cell types and thus unlikely to be uniquely responsive to cell specific physiological stimuli can be deleted from the pool of cDNA, for example, using subtraction hybridization. In addition, proteins whose abundance is altered by the stimulus to be tested may be eliminated from the pool using the subtractive hybridization technique based on the concept that proteins whose level of expression is altered will be unlikely also to experience translocation in response to the stimulus.

In one embodiment, the method of localization is improved by identifying the position of the protein of interest relative to one or more landmarks of cellular location, labeled with unique tags. More specifically, the average distance between tags (landmark vs. protein of interest) is calculated. Such a method for quantifying location is advantageous with respect to known markers as well and is not limited to instances where such markers are to be identified. This method is applicable to intracellular locations of the proteins, as well as to location of proteins displayed on the cell membrane or secreted, since the cell membrane itself may be used as a landmark.

### BRIEF DESCRIPTION OF THE DRAWINGS

FIGS. 1A-1D illustrate an assay for translocation performed by measuring a change in the distribution of distances between tagged copies of a landmark protein and tagged copies of a protein of interest.

FIGS. 2A-2E illustrate shotgun cloning using vectors associated with tags whose identity is readable in situ.

### MODES OF CARRYING OUT THE INVENTION

In the method described, proteins that exhibit translocation in response alterations in the extracellular milieu are coupled to a plurality of distinct labels, which are then tracked to monitor cellular responses to stimuli. In a preferred embodiment, translocating proteins are labeled with a marker that provides a phenotype which can be tracked externally without requiring the cell to be ruptured or otherwise removed from its environment. Preferred examples of markers include particulate labels, such as latex beads or quantum dots and antigenic markers. Translocating proteins can also be constructed as fusion proteins which comprise one or more labels that permit localization of the translocating protein in the cell.

Protein translocation is divided into three general classes. The first class of translocation occurs intracellularly, from one intracellular space to another. An example of intracellular translocation is the movement of pigment organelles in Xenopus laevis, which are used by the animal to change skin color. The second class of translocation occurs when a protein translocates from an intracellular space to an external membrane. Antigen presentation is an example of this class of translocation, where a MHC complex is transported to the surface of a cell to display an antigen. The third class of translocation occurs when an intracellular or membrane bound protein is secreted into the extracellular milieu. For example, T cells are characterized patterns of cytokine secretion in response to antigenic stimulation. Methods for identifying and monitoring proteins that undergo translocation are useful as markers for responses to various stimuli, including drugs, toxins and the like.

The described invention provides methods to identify proteins that are useful as markers for cellular responses whether translocated intracellularly, at the cellular membrane, or extracellularly. In order to identify such markers in an efficient manner, it is desirable to label a multiplicity of protein candidates so that their possible translocation responses to externally imposed conditions can be determined. In order to distinguish the various individual proteins encoded in the genome, unique identifiers for tens of thousands of individual proteins must be prepared or, alternatively, a single or small number of identifiers can be applied to clones distinguished by location (e.g., in different wells of a microplate). One approach to providing the latter type of label is described by Jarvik in U.S. Pat. No. 6,472,207, incorporated herein by reference.

In the Jarvik method, a peptide-encoding segment is inserted into an intron separating exons of an endogenous protein by retroviral transposon insertion. When the nucleotide sequence encoding the protein is expressed, after splicing occurs, the endogenously produced protein will contain an epitope provided by the peptide so that it can be detected by binding to a labeled antibody against that epitope. This technique has been used to assess, among other parameters, the subcellular location of the encoded protein, although alteration of the location as a measure of response to externally provided conditions was not suggested by Jarvik.

A similar approach to providing labels is set forth in an article by Simpson, J. C., et al., EMBO Reports (2000) 1:287-292. In this approach, green fluorescent protein-encoding DNA is coupled either to the N-terminus or C-terminus of particular selected and identified cDNAs that have been amplified in the region of their open reading frames and transfected into host cells. The localization of the resulting fusion protein is used by Simpson, et al., to determine its probable function (e.g., transcription factors are expected to be localized in the nucleus while ion channels are expected to be in cell membrane). Correlations between the conclusions reached using Simpson's localization method as compared to conclusions reached using DNA sequence analysis are significant, but not perfect. In any event, Simpson fails to suggest altering externally imposed conditions to determine the effect of such conditions on the localization patterns.

Another embodiment of the invention relates to the use of reporter gene assays which exploit the presence of a marker fused to an identified translocating protein. Reporter gene assays are useful for studying the impact of perturbations on particular signaling pathways, such perturbation culminating in production of a reporter mRNA or protein. Typical reporters include luciferase and beta-galactosidase. Other suitable reporters include drug resistance factors or enzymes to overcome an auxotrophic deficit. The latter type enables not only observations of the reporter but also selection for its expression.

Typically, DNA encoding a reporter is downstream (i.e., 3′thereto) of a regulatory DNA sequence which in turn is responsive to a particular transcription factor or factors. If the cell state, following perturbation, leads to activation of a transcription factor or factors that drives production of the reporter, then presence of the reporter illuminates mechanism of action of the perturbant. Most commonly, the perturbant is a drug candidate. Other perturbants of interest are secreted proteins of unknown function, antisense or small interfering RNA constructs, or peptides (expressed intracellularly or manufactured by some other means and introduced into the cell by direct permeation or by liposome assisted permeation or similar means).

Secreted proteins as the reporters are preferred since they expand the surface area to which particulate labels can bind, thereby simplifying detection of many such labels. Cell surface antigens are acceptable for the same purpose, however, and even intracellular antigens are useable. A family of epitope-tagged reporters is desirable, but not necessary. Each reporter can be a distinctive analyte (protein or mRNA) to which a specific detector (antibody or complementary DNA) can be prepared and coupled to a distinguishable particulate label.

Epitope tags may be very short peptides or somewhat longer ones as is preferable if the latter are particularly robust. For example, avian pancreatic peptide is a 36 amino acid peptide that is resistant to heat induced unfolding up to 60 degrees C. Variation in exposed residues of the peptide does not affect the stability of the folded peptide, but allows many distinctive peptides to be produced, as described in WO 01/81375, Schepartz, et al.

In one illustrative embodiment, a cDNA library is cloned into expression vectors and transfected into cells of interest. The cells of interest may be any cells, and includes those that may be affected by disease or toxin or other physiological stimuli. Cells of interest may include cells of the immune system, muscle cells, osteoblasts, osteoclasts, specialized hematopoietic cells, stem cells and their differentiated progeny, and the like. Tumor cells are also of interest in light of the desirability of affecting their function therapeutically.

The cDNA inserts in the expression vectors are modified to include a reporter gene construct which comprises nucleotide sequences encoding a reporter sequence and an epitope that is responsive to a detecting agent such as a labeled antibody or a labeled antibody fragment. Suitable epitopes include any short peptide sequence to which antibodies can be prepared or for which a common specific binding partner can be found. Some convenient epitopes commonly used as tags include the FLAG epitope, the c-myc peptide, hexahistidine, and various peptides used as subunit vaccines.

Thus, in this method, the location of a particular expressed protein is determined by the binding of a “detecting agent” to an epitope to which it is fused. The detecting agent is any specific binding partner for the epitope and may be an antibody or immunologically reactive fragments thereof, a chelate, such as is reactive with the hexahistidine tag, or any other moiety known to bind to the selected epitope.

An “epitope” refers to any short peptide sequence for which a specific binding partner can be found. Thus, “epitope” has a broader meaning as used in the present application than that often used in the art—it is not necessarily a peptide which is able to elicit a specific immunological response. It is any segment that may be coupled to a specific binding partner of any kind. The “detecting agent” refers to the specific binding partner which need not be an antibody or a fragment thereof.

The “epitope” is typically a short amino acid sequence, typically containing 20 amino acids or less, more commonly 10 amino acids or less, and preferably 5 amino acids or less. Longer epitopes may optionally be employed; however, it is preferred to use short sequences which are less likely to interfere or alter the pattern exhibited by the protein to which it is fused.

The detecting agent carries with it a label which, in a preferred embodiment, is a fluorescent label or other visible label detectable by wide field or confocal microscopy. In general, a label can be considered to emit, ultimately, a signal which is characteristic of it. The signal is typically electromagnetic radiation either by formation of a colored enzymatic product, by a radioisotope, or by reflection or fluorescence. While enzyme or radioisotope labels could theoretically be used, fluorescent labels such as coupled fluorescein, dansyl, Texas red and the like, or small beads containing one or more such dyes to achieve higher fluorescence intensity, or quantum dots, are more amenable to the practice of the invention. Intrinsically detectable tags such as green fluorescent protein (GFP) could theoretically be used, but it is preferable to use an epitope tag for which high sensitivity detection is feasible thus allowing low level expression of the protein so as not to perturb the normal stoichiometry of protein complexes within the cell that mediate translocation.

The cells are plated or otherwise distributed for inspection by microscopy. Prior to the microscopic examination, the cells are treated with the labeled detecting agent and the location of the detecting agent in the cells is then visualized. A parallel group of cells is then subjected to an external stimulus such as a toxin or other chemical or physiological stimulus. The location of the foreign protein is then, again, observed. Those proteins that exhibit translocation are thus identified as markers for response to the particular stimulus considered.

The intracellular location of candidate proteins indicates translocation and translocation can also occur through display on the cell surface or through secretion. The epitope-detecting agent may thus locate a “halo” of secreted protein surrounding the cells. Various types of proteins are secreted such as autocrine and paracrine factors such as cytokines and matrix metalloproteases.

If all the cells in a particular set of wells of a microplate, or the equivalent, are transformed to express tagged candidate protein #1, under varying stimuli, then the effect of the stimuli on protein #1 is directly defined. Protein #2 can then be examined in a separate set of wells. For a large library of candidate proteins, and a variety of stimuli, the work load quickly becomes burdensome. Accordingly, it is advantageous to perform “shotgun cloning” in which all the cells in a large chamber, such as a Petri dish, are transformed concurrently with numerous different gene vectors at a concentration such that each cell typically receives at most one vector species. If the phenotype of the expressed protein leads to a growth or survival advantage, then the identity of the protein can be determined by recovering the DNA insert from the resulting colony and sequencing. However, for phenotypes such as the ability to undergo translocation, which may not have any impact on growth or survival, a more convenient procedure involves labeling each expression vector with a particulate label identifiable by its unique hue. Such multihued labels are described, for example, in U.S. Pat. No. 6,492,125, incorporated herein by reference. If the DNA is non-covalently associated with the particulate label, then it is feasible to induce uptake of the complex into endosomes, followed by release of the expression vector after transfection into the cytoplasm, while the particulate label remains in the endosome, as disclosed in WO 01/51092. If necessary, each DNA in the library can be pre-sequenced and coupled to a label of known hue. However, more conveniently, if a replicate of the cDNA library and associated particulate labels are available, then the replicates can be used to identify the DNA corresponding to the particulate label of interest. For example, automated colony pickers can be used to distribute bacterial clones into the wells of 1536 well plates, beads of a single hue added to each well in a manner that allows capturing the vector DNA, and a sampling of all the wells used for the transfection experiment. In this manner, many stimuli can be applied, cell numbers used can be minimized and the overall efficiency increased.

In somewhat more detail, a library of expression vectors for fusion proteins is introduced into cells using standard transduction techniques. The fusion proteins in the library are composed of candidate potentially translocatable proteins fused to an epitope detectable by a common detecting agent. The expression vectors also are associated non-covalently with a detectable label wherein the signal emitted by each label is indicative of the particular expression vector with which it is associated. (The library must have been prepared in advance using known nucleic acid components which are then associated with a distinctive label, alternatively, only the relevant DNA members of the library need be sequenced using the replication technique described above.) The treated cells are then plated so that individual cells or colonies are observable and the nature of the fusion protein generated identifiable by the distinctive signal of the label. Replicates of the array are then used to observe the behavior of the fusion proteins by treating the cells with the detecting agent, observing the location of the detecting agent bound to the protein, treating the cells with a stimulus such as a toxin or drug and then observing the location of the detecting agent after stimulation. The location can be simply observed or more accurately quantitated by the relationship of the detecting agent to a labeled landmark as further described below. Those fusion proteins that exhibit translocation, either within the cell or by being secreted, are then identified as suitable translocation markers. The nature of the protein that is the marker is preferably pre-identified by virtue of the “shotgun cloning” technique described above.

The above-described method of “shotgun cloning” of a multiplicity of cDNA or other nucleic acid molecules is useful in other contexts as well as in the claimed process for identification of translocation markers. Any work wherein it is desired to know the nature of a nucleic acid introduced into a cell in an efficient manner may employ this technique. For example, it may be desirable to screen a cDNA library for a gene that encodes a protein that functions as a cell surface receptor that can be detected by an appropriate antibody or labeled ligand. Cells that contain the nucleic acid encoding the appropriate protein can be identified through this phenotypic characteristic. The nature of the nucleic acid that has successfully transformed this cell can then be ascertained by the hue of the label to which it was attached and which remains in the cell. The advantage to the method is that the association of individual identified DNAs with distinguishable particulate labels can be performed just once on a large batch of a single cDNA (for these purposes, even a few microliters is sufficient to hold tens of millions of the particulate labels which are on the order of 1 micron in diameter). That reagent pool can then be used for many different experiments.

One of the advantages of the shotgun cloning method of the invention is that multiple transformations can be evaluated simultaneously since cells in mixtures can be distinguished by color or hue of the associated particulate label. Thus, in a single well with a size sufficient to accommodate 1,000 cells, each of which can be viewed individually microscopically, a mixture of, for example, 50 DNAs each labeled with a label of a characteristic hue may be employed. The quantity of cDNA in the mixture is sufficiently low that it is unlikely that more than one DNA will be taken up by a single cell. Thus, using a Poisson distribution as a rough approximation, a population of about 10 cells should contain at least one and more typically several cells with a single copy of a particular cDNA; all 50 cDNAs in the mixture would be similarly represented and distinguishable by the label carried into the cell, again using microscopic techniques. The microscopic evaluation can be employed with respect to cells in suspension as well as cells that are adherent to the surface of the well. Since 50 cDNAs can be evaluated in a single well of a 96-well plate, employing all of the wells results in simultaneous ability to evaluate the effect of various conditions on the translocation of proteins encoded by almost 5,000 cDNAs. Surveying the entire genome is thus feasible.

Evaluation of other characteristics could also be employed using cells modified according to this technique. Thus, for example, the ability of the cells to resist cancer chemotherapeutic agents as described in U.S. Pat. No. 6,326,448 could be evaluated using this platform. The response of cells to conditions that do not confer a growth advantage can also be evaluated since it will not be necessary to isolate the transforming DNA from the cells exhibiting a phenotypic characteristic. Phenotypes of interest include secretion of particular proteins, such as cytokines, which can be assayed in situ using an ELISPOT assay. Surface expression of antigens can likewise be assayed in response to the introduced protein.

It is not, of course, necessary to employ a 96-well plate. The size of the well will determine the number of cells that can be, in practice, evaluated; if a greater population of cells is supplied in a larger well, then a greater multiplicity of cDNAs labeled with their particulate distinctive labels could be used per well.

The DNA can be supplied to the cells coupled to the particulate labels using uptake mechanisms including endocytosis as described in PCT publication WO 99/07414. In this method, vectors derived from antibodies are employed to target the endosomes. This is described as well in a paper by Avrameas, A., et al., Proc. Natl. Acad. Sci. USA (1998) 95:5601-5606. The release in endosomes is facilitated by hydrophilic/hydrophobic coupled polymers that act as membrane barrier transport enhancing agents and become membrane disrupting after endocytosis. The polymers are coupled to nucleic acids through linkages modified by the low pH of endosomes as described in PCT publication WO 01/51092.

Thus, in general, shotgun cloning can be accomplished in this and other contexts by coupling each member of a DNA library to a particulate label of a particular hue whereby the presence of the particular hue in a cell or cell colony indicates the presence of the already identified and, typically sequenced, DNA.

It is possible to use generic cDNA libraries or other mixtures of DNA encoding multiplicities of proteins to identify useful translocation markers encoded thereby. Thus, to identify translocation markers that are operative in hematopoietic cells, cDNA obtained from hematopoietic cell lines are a preferred source for the library. For assessing translocation in osteoblasts, for example, cDNA libraries from this type of cell are preferable. Use of cDNA libraries derived from particular types of cells of interest is not a requirement, but would have a higher probability of yielding meaningful results than arbitrarily chosen cDNA libraries.

An additional approach to enhancing the probability of finding a multiplicity of suitable markers is to employ mixtures of nucleotide sequences that encode classes of proteins known to exhibit translocation responses in the presence of various stimuli. Examples of such classes include the protein kinase C family which contains at least 10 known members as described by Mochly-Rosen, D., et al., FASEB J (1998) 12:35-42; cAMP-dependent kinase anchoring proteins (AKAP) with about 50 known members at present as described by Pawson, T., et al., Science (1997) 278:2075-2080 and by Pawson, T., et al., Genes & Development (2000) 14:1027-1047; and pleckstrin homology domain proteins (PH) with about 200 known members as described by Lemmon, M. A., et al., FEBS Lett. (2002) 513:71-76. Examples of secreted proteins that would be useful in this context include the various cytokines, growth factors, hormones, and the like.

Other methods to enhance the probability of success rely on eliminating unlikely candidates, such as housekeeping genes. These genes can be identified (actually removed from consideration) using subtractive hybridization techniques as described in U.S. Pat. No. 5,589,339 incorporated herein by reference. That is, mRNAs that are found in a multiplicity of cell types are subtracted from mRNA pools, thus identifying members of these pools that are unique to a cell type of interest. It is believed that housekeeping genes, common to many cell types, are less preferred embodiments of markers for translocation. Similar techniques can be used to eliminate mRNA sequences that are elevated in abundance under externally imposed conditions on the theory that where expression levels are changed, translocation of the expressed protein is unlikely to occur.

Another aspect of the invention that is useful in identifying markers, but also in other contexts, is the assigment of location landmarks using particulate markers. Conventional staining dyes and conventional immunostaining provide only limited data channels that can be used to define such landmarks. Further, the continuous nature of the stained images requires that an artificial procedure be applied to prepare the image for cross-correlating two distributions (introduction of “grain” into the continuous distribution).

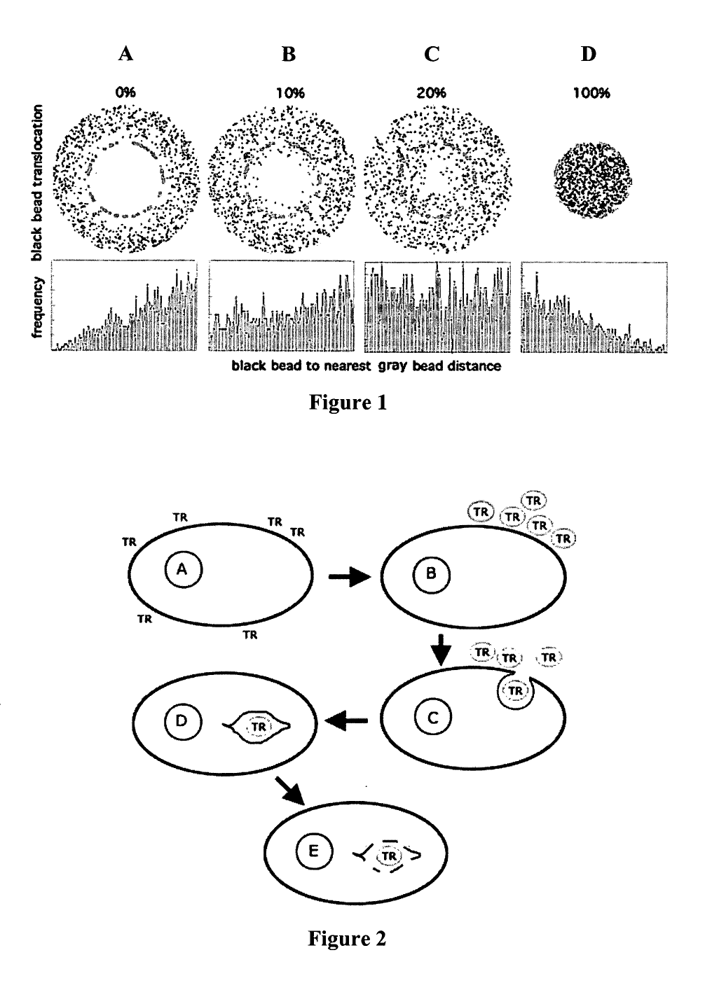

FIG. 1, as described in more detail below (Example 1), illustrates the idea of defining location relative to a landmark, measured using particulate labels that provide “grain” naturally. For all these reasons, the use of particulate labels is preferred. Thus, the location of any arbitrary protein or other labeled material intracellularly is evaluated relative to particular landmarks, typically denoting major organelles for example, as compared to a generalized assignment to a 3-D position in space within the cell. Location of proteins or other molecules of interest relative to the nucleus, mitochondria, cytoplasm, or other subcellular structures can then be evaluated.

Once again, the particulate labels described in U.S. Pat. No. 6,492,125 referenced above provide sufficient data channels for marking multiple landmarks. The ability to provide particulate based labeling of subcellular location points is thus a substantial advantage in identifying the intracellular movement of candidate marker proteins and the use of location markers in this context is an aspect of the present invention, even if the distinction between locations is accomplished by conventional techniques such as those disclosed in U.S. Pat. No. 5,989,835. However, the improvement wherein the intracellular signposts are labeled by particulate, granular labels is useful and an aspect of the invention in a larger variety of contexts.

For example, as disclosed in Porter, A. G., Trends Cell Biol. (1990) 9:394-401, the sequence of events which results in apoptosis in T cells is characterized by a number of translocations which can be followed using this method. The initiation of apoptosis is marked by translocation of Bax to the mitochondria, followed by leakage of cytochrome P450 from the mitochondria. Still later, caspase-cleaved substrates translocate to the nucleus, each exhibiting a particular translocation pathway.

Labeling the various intracellular organelles, such as the mitochondria, nucleus, and additional marker proteins of known location, with specific hued beads, makes following this process simpler than would be possible using non-particulate labels for the signposts. In addition, the cell membrane itself may be labeled as a particularly useful signpost for ascertaining the translocation of surface displayed and secreted proteins.

In another example, it is known that a fluorescent derivative of the drug, phalloidin binds to actin filaments and can be used as a label for these filaments. Migration of neutrophils toward a chemotactic factor is evidenced by polarization of the actin cytoskeleton toward a pipette delivering the factor as monitored with the labeled actin. The change in direction of actin polarization when the location of the chemotactic factor is altered can be followed quantitatively by computing the vector sum of all of the actin fragments, enabling monitoring of changes that occur much earlier than is visible to a human observer. The ability to observe and analyze individual vectors is improved by the use of particulate labels as opposed to the continuous phalloidin label previously employed.

As described in U.S. application Ser. No. 09/332,611, which is now published as PCT publication WO 00/77517, the pattern of localization of various proteins can be used to characterize the condition of cells and the effects of a wide variety of perturbants that influence cell condition. Use of the markers identified by the method of the invention and use of the improved localization method involving particulate labeled signposts is particularly advantageous in conducting these methods and in evaluating the effect of protocols and individual compounds on them.

Screening for drugs that interfere with protein localization, such as described in U.S. Pat. No. 5,935,803 incorporated herein by reference, is also facilitated by the improved methods for quantifying location disclosed herein. Examples of translocating protein families suitable for such drug screening include but are not limited to PKC, AKAP, PH, Bax/Bcl, SH2 and SH3 domain proteins, steroid receptors, cytokines, hormones, leucine zipper and zinc finger transcription factors. Screening for specific translocating proteins identified as correlated with specific physiological states are more particularly enabled.

The following examples are offered to illustrate but not to limit the invention.

### EXAMPLE 1

### Simulated Particulate Labeling for Measuring Nuclear Translocation

Nuclei can be labeled using conventional dyes, such as the intercalating fluorescent dye DAPI, or by immunostaining, e.g., using antibody to the nuclear pore complex at the boundary of the nucleus or to histones inside the nucleus. Similarly, proteins that translocate to the nucleus, such as NF-kB or steroid receptors, can also be labeled with antibody. If the antibody tags are particulate, then even a small number are sufficient to enable measuring changes in the distribution of such translocating proteins relative to the landmark proteins defining the nucleus. In the illustrated example, 1000 (black) beads are used to define the translocating protein, and 100 (gray) beads are used to define the nuclear envelope. The distance from each (black) bead to the nearest (gray) bead is measured, and the frequency of different bead-to-bead distances plotted. As the percentage of beads that have translocated increases, the histogram changes markedly. This example is used to illustrate a typical situation. With the nucleus defined by histone labeling, the results will be even more pronounced. If the cell perimeter is also labeled, then the ratio of distances between a (black) bead and the nuclear marker vs. the perimeter marker can be used to provide even more robust discrimination of translocation.

The advantage of measuring bead-to-bead distances is that only a small fraction of the landmark and translocating antigens need to be labeled, thus simplifying inclusion of multiple landmark and translocating antigens in a single assay. With conventional staining, a much higher fraction of the antigens must be labeled in order to generate strong enough signal for detection and an arbitrary process for introducing grain must be imposed in order to compute a parameter analogous to median nearest neighbor distance. This advantage applies to any particulate label with a sufficiently strong signal for single particle detection, including the particles described in U.S. Pat. No. 6,492,125, incorporated herein by reference, as well as quantum dots, metal clusters, and nanobar codes.

As shown in FIGS. 1A-1D, the histogram of distances between marker and label changes dramatically as translocation of the black bead labeled protein from the cytoplasm to the nucleus occurs. The nuclear envelope is labeled with gray beads and the frequency distribution of distances between black and gray beads is plotted as the translocation occurs.

As shown in FIG. 1A, when translocation has not yet occurred, a larger number of black beads are separated at high distances from gray beads than are in close proximity to gray beads. As shown in FIG. 1B, when about 10% of the black bead labeled proteins have translocated, the frequency distribution of the black bead to nearest gray bead distance flattens and this flattening continues in FIG. 1C where 20% of the translocated protein has entered the nucleus. In FIG. 1D, with 100% of the translocated proteins in the nucleus, the highest frequencies of distance between black beads to nearest gray bead are in the shorter distance portion of the histogram.

### EXAMPLE 2

### Shotgun Cloning

In this example, a library of cDNA, each individual cDNA labeled with a distinctive tag, is used to transform cells. The identity of the transforming DNA is ascertained by the characteristic label to which it is attached. As illustrated in FIG. 2, the label is released from the DNA that has been introduced into the cells, permitting the DNA to function without encumbrance from the identifying label. The introduction of the DNA is enhanced by the availability of transferrin receptor which occurs on the surface of many cells. FIG. 2 illustrates this process.

Panel A shows a cartoon of a single cell having transferrin receptor (TR) on its surface. Panels B and C show that TR aggregates and is endocytosed when treated with an antibody against TR, where the antibody may be associated with a particulate label. Panel D assumes DNA is also associated with the particulate label; the uptake of TR will also lead to uptake of the particulate label and DNA into the endosomal compartment. Panel E assumes that the particulate label includes agents that destabilize the endosome integrity so that the DNA is released into the cell, where it can direct production of the encoded protein. The particulate label remains in the damaged endosome, allowing identity of the released DNA to be determined.

### EXAMPLE 3

### Multiplexed Reporter Gene Constructs

T-lymphocytes regulate cytokine production by several parallel pathways mediated by transcription factors such as NF-κB, NFAT (nuclear factor of activated T cells), and AP-1. Three expression systems are constructed. Each expression system comprises a promoter which is regulated by NF-κB, NFAT, or AP-1; a reporter gene, which is luciferase for all three systems, and three distinct epitope labels which are specifically recognized by three different monoclonal antibodies.

The expression systems are introduced into a population of T-lymphocytes, and the cells are distributed into individual wells on an assay plate. Luciferin, the substrate for luciferase, is present in each well. A plurality of candidate expression regulators or stimuli is also provided to the wells of the plate at known locations. Detection of luciferase activity in a well indicates transcriptional activity in response to the candidate regulator. In those wells, the distinct monoclonal antibodies and then exposed to the contents of the well to determine which promoter is activated by which candidate regulator or regulators.

### EXAMPLE 4

### Immunogenic Reporter Gene Constructs

A reporter gene construct is prepared comprising a reporter gene, a secretion sequence, and an epitope tag. Specifically, the reporter gene is the secreted form of alkaline phosphatase (SEAP), which lacks a transmembrane sequence and thus permits the enzyme to be secreted from a cell containing the reporter gene construct. The construct also comprises an epitope tag such as the myc peptide. In Example 3, luciferase activity is used to monitor gene expression in response to a stimulus that upregulated a particular promoter. In this example, detection of the secreted protein serves the same function as detection of luciferase. By varying the epitope tag such that each tag is recognized by a specific detecting agent, multiple reporter constructs can be studied in the same cell, allowing non-linear interactions among signaling pathways to be discovered and analyzed. One reporter, driven by NF-kB, uses the FLAG peptide as the epitope. Many such epitope tags can be created, and detection of them can be accomplished even for single cells by using antibodies linked to particulate labels that can be prepared in many distinguishable types, as described in U.S. Pat. No. 6,642,062. Accordingly, the efficiency of reporter gene assays is improved.

1. A method to identify at least one protein translocated in response to a stimulus, comprising:
applying said stimulus to a multiplicity of cells, each cell expressing a fusion protein,
wherein each fusion protein comprises an amino acid sequence ordinarily found in the cell in which it is expressed fused to an epitope, wherein said epitope binds to a detecting agent common to the epitopes present on all fusion proteins in all cells;
providing the cells with said detecting agent;
observing localization of the detecting agent before and after application of said stimulus; and
comparing the location of each fusion protein before and after said applying;
wherein a protein whose location is altered after said applying as compared to before said applying is identified as a translocation marker for said stimulus.

2. The method of claim 1, wherein the epitopes fused to each amino acid sequence are identical.

3. The method of claim 1, wherein in said observing step, the cells are displayed as an array.

4. The method of claim 1, wherein said translocation marker is translocated intracellularly.

5. The method of claim 1, wherein said translocation marker is translocated so as to be displayed on the cellular membrane.

6. The method of claim 1, wherein said translocation marker is translocated across the cell membrane and secreted.

7. The method of claim 1, wherein said fusion proteins are produced from a library of nucleic acids each member of which has been previously sequenced and labeled with a particulate label, wherein said particulate label has a hue distinctive for the nucleic acid which it labels.

8. The method of claim 7, wherein said identifying comprises detecting the hue of the label.

9. The method of claim 1, wherein said fusion proteins are produced from a library of nucleic acids each member of which is previously labeled and wherein said library is supplied as an array contacting said multiplicity of cells in a predetermined pattern.

10. The method of claim 4, wherein the location of said fusion proteins is determined in reference to labeled intracellular landmarks.

11. The method of claim 10, wherein the intracellular landmarks are labeled with particulate labels.

12. The method of claim 5, wherein the location of said fusion proteins is determined in reference to a labeled cellular membrane

13. The method of claim 12, wherein said cellular membrane is labeled with particulate labels.

14. The method of claim 6, wherein the location of said fusion proteins is determined in reference to a labeled cellular membrane

15. The method of claim 14, wherein said cellular membrane is labeled with particulate labels.

16. The method of claim 10, which comprises calculating the distance between a label associated with a translocating protein and the nearest label associated with a landmark cellular component.

17. The method of claim 12, which comprises calculating the distance between a label associated with a translocating protein and the label associated with the cellular membrane.

18. The method of claim 14, which comprises calculating the distance between a label associated with a translocating protein and the label associated with the cellular membrane.

19. The method of claim 1, wherein said observing comprises calculating the population distribution of distances between said detection agent and a landmark cellular component.

20. The method of claim 1, wherein said fusion proteins are enriched in proteins known to exhibit translocation.

21. The method of claim 1, wherein said fusion proteins are enriched in non-housekeeping proteins.

22. A method to modify a multiplicity of cells each with a different nucleic acid, which method comprises transfecting said cells with a mixture of nucleic acids, each different nucleic acid in said mixture being associated with a label, wherein the label emits a signal distinctive for the nucleic acid to which it is associated.

23. The method of claim 22, wherein the identity of any nucleic acid in the mixture with respect to the signal has been determined.

24. The method of claim 22, wherein cells are transfected from a library of nucleic acids each member of which is previously labeled and wherein said library is supplied as a predetermined array by contacting said multiplicity of cells with said predetermined array.

25. The method of claim 23, which further includes diluting the mixture of transfected cells into individual colonies and determining the nature of the transfected nucleic acid by identifying the signal emitted by the label.

26. The method of claim 24, which further includes correlating the position of transfected cells with the position of a labeled nucleic acid in the array.

27. A method to visualize subcellular structure, which method comprises coupling each subcellular structure to be visualized with a label wherein said label emits a signal selected to be characteristic of said subcellular structure.

28. A method to detect any effect of a regulator on gene expression, comprising:
providing at least one candidate regulator to a multiplicity of expression systems, wherein each expression system comprises a promoter operably linked to a distinct label that binds to a corresponding detecting agent;
providing the multiplicity of expression systems with said corresponding detecting agents; and
detecting any label produced with the corresponding detecting agent before and after providing said candidate regulator is provided.

29. The method of claim 28, wherein the multiplicity of expression systems is present in a single cell.

30. The method of claim 28, wherein the multiplicity of expression systems is present in a multiplicity of cells.

31. The method of claim 28, wherein each promoter of each expression system is responsive to a distinct transcription factor.

32. The method of claim 28, wherein each promoter of each expression system is responsive to the same transcription factor.

33. The method of claim 28, wherein the expression system further comprises a reporter sequence selected from the group consisting of a secreted reporter, a cell surface reporter, and an intracellular reporter.

34. The method of claim 33, wherein the reporter is a luciferase.

35. The method of claim 33, wherein the reporter is a secreted form of alkaline phosphatase (SEAP).
